(19) United States
(12) Patent Application Publication
(10) Pub. No.: US 2007/0066988 A1
(43) Pub. Date: Mar. 22, 2007

(54) Trocar obturator with cutting edges

(76) Inventors: Keshava Datta, Pasadena, CA (US); Thomas A. Gilker, Cincinnati, OH (US); Michael D. Cronin, Cincinnati, OH (US)

Correspondence Address: WELSH & FLAXMAN LLC, 2000 DUKE STREET, SUITE 100, ALEXANDRIA, VA, 22314, US

(21) Appl. No.: 11/232,226

(22) Filed: Sep. 22, 2005

(51) Int. Cl. A61B 17/34

(52) U.S. Cl. 606/185

(57) ABSTRACT

A trocar obturator includes a shaft having a proximal end and a distal end. The trocar obturator also includes a tip positioned at the distal end of the shaft, the tip including a distally extending blade structure adapted to reduce penetration forces required during insertion of the trocar obturator. The blade structure includes a first cutting edge and a second cutting edge.

### BACKGROUND OF THE INVENTION

1. Field of the Invention

The invention relates to trocar obturators. More particularly, the invention relates to an obturator tip for a trocar obturator which is designed to reduce the required penetration forces.

2. Description of the Prior Art

A trocar assembly is a surgical instrument used to gain access to a body cavity. A trocar assembly generally comprises two major components, a trocar sleeve, composed of a trocar housing and a trocar cannula, and a trocar obturator. The trocar cannula, having the trocar obturator inserted therethrough, is directed through the skin to access a body cavity. Once the body cavity is accessed, laparoscopic or arthroscopic surgery and endoscopic procedures may be performed.

In order to penetrate the skin, the distal end of the trocar cannula is placed against the skin. A cutting blade is then actuated and the trocar obturator is used to penetrate the skin and access the body cavity. By applying pressure against the cutting blade and the proximal end of the trocar obturator, the cutting blade and the sharp point of the obturator are forced through the skin until it enters the body cavity. The trocar cannula is inserted through the perforation made by the trocar obturator and the trocar obturator is withdrawn, leaving the trocar cannula as an access way to the body cavity.

The proximal end portion of the trocar cannula is typically joined to a trocar housing that defines a chamber having an open distal end portion in communication with the interior lumen defined by the trocar cannula. A trocar obturator, or other elongated surgical instruments or tools, axially extend into and are withdrawn from the trocar cannula through the proximal end portion of the chamber defined by the trocar housing.

Current trocar obturators have distal ends with very basic penetration structures. Referring to FIGS. 2 and 3, the common prior art tip design includes a pointed tip and cutting blade extending through the obturator tip in a manner which substantially bisects the pointed tip. This design requires that the surgeon apply substantial force in penetrating the skin of the patient. Typically, penetration forces are approximately 10 lbs for 5 mm trocar obturators and 15 lbs for 12 mm trocar obturators.

With the application of substantial force comes disadvantages due to unnecessary trauma and potential device malfunction. For example, the substantial force required in the use of current trocar obturators, results in great acceleration of the trocar obturator as it passes through the skin of the patient. This, in turn, results in uncontrolled penetration that can ultimately lead to trauma, such as, damage to internal organs.

The prior art has attempted to remedy this situation by employing various tip designs. For example, the angle of the cone at the tip of the trocar obturator has been adjusted and the width of the cutting blade at the tip of the trocar obturator has similar been varied. However, these attempts have been met with only limited success.

As such, those skilled in the art will appreciate that an improved tip is needed which decreases the required penetration forces. The present invention provides a trocar obturator with such a tip.

### SUMMARY OF THE INVENTION

It is, therefore, an object of the present invention to provide a trocar obturator including a shaft having a proximal end and a distal end. The trocar obturator also includes a tip positioned at the distal end of the shaft, the tip including a distally extending blade structure adapted to reduce penetration forces required during insertion of the trocar obturator. The blade structure includes a first cutting edge and a second cutting edge.

It is another object of the present invention to provide a trocar obturator wherein the first and second cutting edges are offset.

It is also an object of the present invention to provide a trocar obturator wherein a secondary flat point angle is positioned between the first and second cutting edges.

It is a further object of the present invention to provide a trocar obturator wherein the first and second cutting edges respectively include a negative cutting angle

It is also another object of the present invention to provide a trocar obturator wherein the first and second cutting edges range from −60° and 0°.

It is still another object of the present invention to provide a trocar obturator wherein the first and second cutting edges range from −45 and −30°.

It is a further object of the present invention to provide a trocar obturator wherein the first and second cutting edges respectively include a positive cutting angle.

It is still a further object of the present invention to provide a trocar obturator wherein the first and second cutting edges range from 0° to 70°.

It is also an object of the present invention to provide a trocar obturator wherein the first and second cutting edges range from 0° to 60°.

It is another object of the present invention to provide a trocar obturator wherein the tip has a generally conical construction.

It is a further object of the present invention to provide a trocar obturator wherein the tip has a cone angle of approximately 30° to approximately 150°.

It is also an object of the present invention to provide a trocar assembly including a trocar sleeve and a trocar obturator shaped and dimensioned for movement within the trocar sleeve. The trocar obturator includes a shaft having a proximal end and a distal end, a tip positioned at the distal end of the shaft, the tip including a distally extending blade structure adapted to reduce penetration forces required during insertion of the trocar obturator, and the blade structure includes a first cutting edge and a second cutting edge.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a perspective view of a trocat in accordance with the present invention.

FIGS. 2 and 3 show a conventional prior art obturator tip design.

FIGS. 4, 5 and 6 show an obturator tip construction in accordance with the present invention.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as the basis for the claims and as a basis for teaching one skilled in the art how to make and/or use the invention.

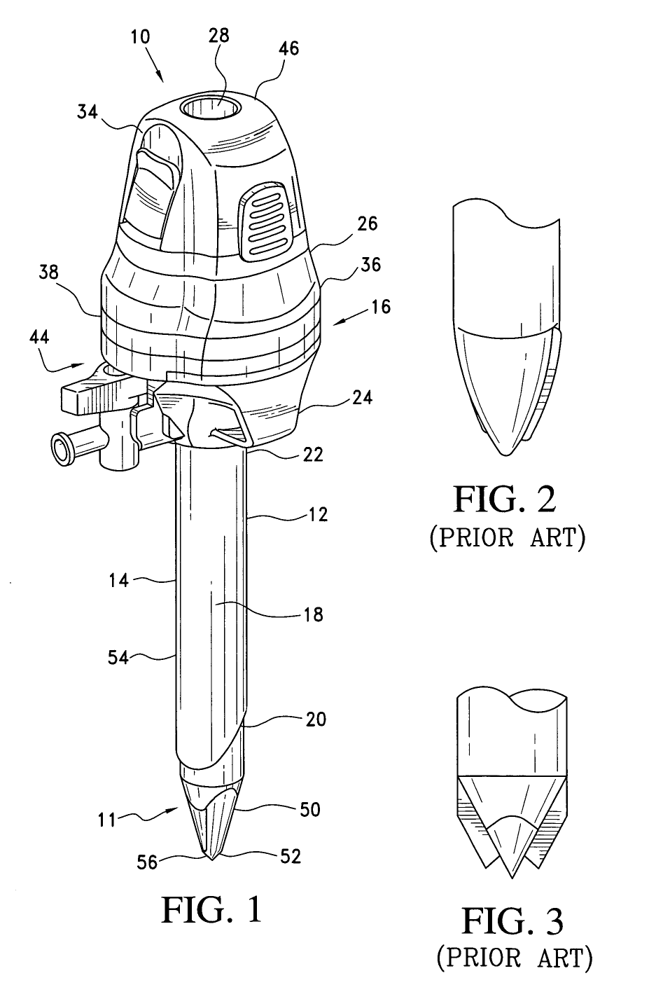

Referring to FIGS. 1, 4, 5 and 6, a tip structure 11 for a trocar obturator 14 is disclosed. The tip structure 11 provides for improved operation of the trocar obturator 14 as it is passed through the trocar cannula 12, trocar housing 16 and patient tissue. As those skilled in the art will certainly appreciate, the concepts underlying the present invention may be applied to a variety of trocar obturator structures without departing from the spirit of the present invention.

Referring to FIG. 1, the trocar assembly 10 generally includes a trocar cannula 12, a trocar obturator 14, and a trocar housing 16. For example, the present trocar obturator is designed for use with a trocar assembly such as that disclosed in U.S. patent application Ser. No. 10/943,222, entitled “ROTATIONAL LATCHING SYSTEM FOR A TROCAR”, filed Sep. 17, 2004, which is incorporated herein by reference. However, those skilled in the art will appreciate the present trocar obturator may be used with a variety of trocar assemblies without departing from the spirit of the present invention.

Briefly, the trocar cannula 12 defines an interior lumen 18 having an open distal end portion 20 and an open proximal end portion 22. The proximal end portion 22 extends into and is mounted in the distal end portion 24 of trocar housing 16. The trocar housing 16 has an open proximal end portion 26 that defines an opening 28. The opening 28 is provided with a proximal seal assembly (not shown). The opening 28 is further provided with a duckbill seal assembly (not shown) positioned beneath the proximal seal assembly.

In general, the trocar sleeve 44 is composed of a trocar cannula 12 and a trocar housing 16. The trocar housing 16 includes a first housing member 36 and a second housing member 38. Although, the housing 16 is disclosed as two components it is contemplated that a single component could be used without departing from the spirit of the present invention. The two component housing shown, aids in removal of specimens.

The trocar obturator 14 is sidable in and removable from within the trocar cannula 12 and is inserted into the trocar housing 16 and the trocar cannula 12 through the proximal seal assembly, the duckbill seal assembly and the opening 28 of the trocar housing 16. An obturator handle 34 is provided at the proximal end 46 of the trocar obturator 14 and a point or blade is formed at the distal end 50 thereof. As is well known in the art, the proximal seal assembly cooperates with the exterior of the instruments (for example, trocar obturators and other tools adapted for use in conjunction with trocar based procedures) extending through the trocar sleeve 44 to sealingly engage the exterior surface thereof and thereby preclude the passage of fluids through the trocar housing 16.

Referring to FIGS. 1, 4, 5 and 6, the trocar obturator 14 in accordance with a preferred embodiment of the present invention will now be described in greater detail. The trocar obturator 14 includes a proximal end 46 to which a handle 34 is secured. The trocar obturator 14 further includes a distal end 50 including a tip member 52 forming the focus of the present disclosure. In accordance with a preferred embodiment of the present invention, the tip member 52 is made from polycarbonate, however, those skilled in the art will appreciate that other materials may be used without departing from the spirit of the present invention. Between the distal end 50 and the proximal end 46 of the trocar obturator 14 is a shaft 54 that connects the tip member 52 to the handle 48.

With particular reference to the distal end 50 of the trocar obturator 14, the tip member 52 includes a distal tip construction optimized for reducing the penetration forces required during insertion of a trocar obturator 14. The distal tip 56 construction also provides for improved visibility when a camera is used in conjunction with the trocar obturator 14, as the tip member 52 may be formed of clear materials allowing viewing therethrough. Better viewing is a result of the flat angle formed at the center of the tip member 52 of the trocar obturator 14. With this in mind, the present tip construction allows for the creation of a flat angle at the center of the tip member 52 of the trocar obturator 14, thereby allowing for improved viewing therethrough.

As will be discussed below in substantially greater detail, the key factor governing the optimization of the tip member 52 of the trocar obturator 14 is the geometry at the distal tip 56, which controls the torque and thrust forces required during penetration. In fact, the majority of penetration force is controlled by the tip member 52, and particularly, the distal tip 56, as it separates the layers of tissue during penetration.

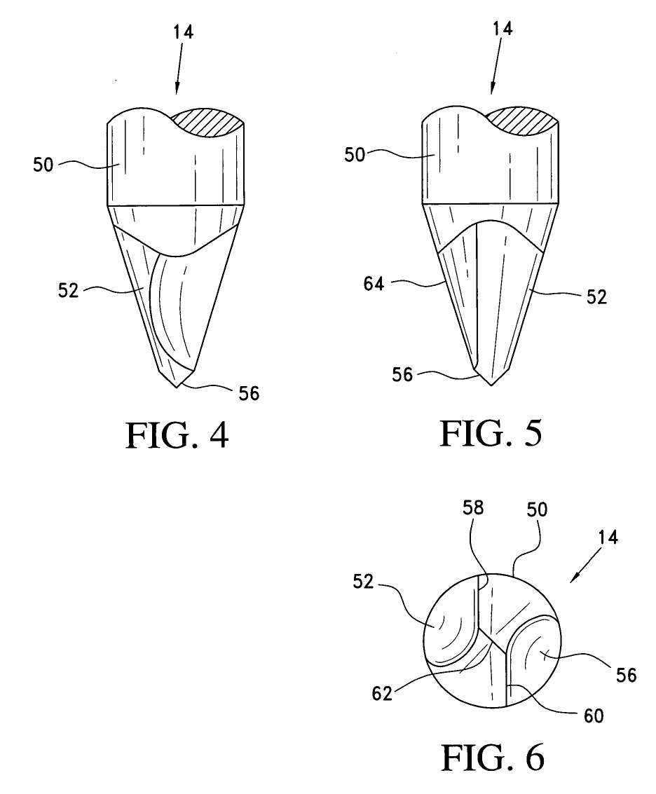

In accordance with a preferred embodiment of the present invention, and with reference to FIGS. 4, 5 and 6, the geometry of the distal tip 56 is optimized through the inclusion of offset cutting edges 58, 60 with a cutting angle and a secondary flat point angle 62 at the center. In addition, the secondary flat point angle 62 at the center provides for improved vision through the trocar obturator tip member 52 by centering the field of vision to achieve greater focus.

As those skilled in the art will certainly appreciate, the cutting edges are formed in a manner similar to cutting edges found in traditional drill bits. As such, each of the cutting edges may generally be thought of as being composed of opposed surfaces that meet at a substantially sharp point. While a pair of cutting edges are disclosed in accordance with a preferred embodiment of the present invention, it is contemplated the tip member may have multiple cutting edges, for example, three or four. The idea is to break the cutting blade into a number of smaller edges with optimized angles based on thrust forces encountered during penetration. Further, and with regarding to the flat point angle, it is contemplated this need not be flat, but may be anywhere from 130 to 180 degrees without departing form the spirit of the present invention.

The distal tip 56 of the present trocar obturator 14 includes a primary cone 64, which is defined as the portion of the distal tip 56 which tapers in from the shaft 54 of the trocar obturator 14 toward the offset cutting edges 58, 60. In accordance with a preferred embodiment of the present invention, the primary cone 64 is preferably formed at an angle of approximately 30° to approximately 150°, and more preferably, 30° to approximately 35°, with reference to the central axis of the trocar obturator 14.

Distal to the primary cone 64 are the offset cutting edges 58, 60 and the secondary flat point angle 62. The offset cutting edges 58,60 are substantially mirror images of each other and are oriented to be substantially parallel to each other. Connecting the offset cutting edges 58, 60 is the secondary flat point angle 62 which extends between the first and second cutting edges 58, 60 through the central axis of the trocar obturator 14. Although two cutting edges are disclosed in accordance with a preferred embodiment of the present invention, additional cutting edges may be employed, for example, 2 to 6 cutting edges, without departing from the spirit of the present invention.

As will be discussed below in greater detail, each of the cutting edges 58, 60 may be provided with a variety of shaped cutting edges within the spirit of the present invention. Each of the cutting edges 58, 60 may be provided with a positive cutting angle in the range of approximately 0° to approximately 70°, more preferably, approximately 0° to approximately 60°. The cutting edges may also be provided with a negative cutting angle in the range of approximately −60° to approximately 0°, more preferably, between approximately −30° and approximately 45°. As those skilled in the art will certainly appreciate, the ultimate cutting angle employed will depend upon the application for which the trocar obturator is designed and may be varied without departing from the spirit of the present invention.

With regard to the specific geometry employed in the construction of the offset cutting edges 58, 60 with a cutting angle and a secondary flat point angle 62 at the center, these components are optimized by adjusting the torque and force thrust (that is, the normal force applied during penetration) to generate an ideal penetration force. In particular, the problem may be stated as:
Tₜₒₜₐₗ=Constant−Reduce Fₙ based on geometry.

where,
Tₜₒₜₐₗ=total torque applied to the trocar obturator during penetration
Fₙ=normal force applied during penetration
Constant=total penetration force.

Optimization through consideration of this equation is possible with the offset cutting edges 58, 60 used in accordance with the present invention. Ideally, the penetration force should be approximately 10 lbs with the Tₜₒₜₐₗ and Fₙ adjusted to achieve desirably results. It has been found in certain applications that when the cutting angles are set aggressively from approximately 40° to approximately 60° one may readily optimize the penetration forces. In addition, the resulting obtuse secondary cone angle provides better visibility at the center of focus.

In accordance with a preferred embodiment of the present invention, the tip is constructed to provide for approximately 90 degrees to approximately 270 degrees, and most preferably approximately 150 degree, motion of torque while inserting the trocar obturator 14. With regard to the thrust force required in accordance with a preferred embodiment of the present invention, the force is set to be approximately 1 in-lbs for a 5 mm trocar obturator and at approximately 3.8 in-lbs for a 12 mm trocar obturator. While thrust forces are present above in accordance with a preferred embodiment of the present invention, the goal in the development of the present is minimizing thrust forces and the specific thrust forces may be varied without departing from the spirit of the present invention.

The cutting edges 58, 60 form the major element of the distal tip 56. The cutting edges 58, 60 are offset and formed with a predetermined cutting angle optimized for performance in accordance with the present invention. The distal tip 56 also includes a secondary flat point angle 62 connecting the offset cutting edges 58, 60. The cutting edges 58, 60 are responsible for cutting and separating the tissue through which the trocar obturator 14 passes.

In particular, the dynamic cutting angle (α_{dyn}) of the respective blades of the offset cutting edges 58, 60 employed in accordance with the present invention is measured in a plane through a point on the respective cutting edge 58, 60 and perpendicular to the horizontal line that passes that point and intercepting with the trocar obturator center axis, between the cutting face and normal line of that plane which contains both the cutting edge 58, 60 and the cutting velocity vector. The cutting velocity vector is the vector sum of the rotary cutting velocity vector and the feed velocity vector. That is, the dynamic cutting angle of the distal tip 56 in accordance with the present invention based upon the normal and rotary forces applied by the distal tip 56 during penetration of the trocar obturator 14.

Referring to the formula presented below, adjusting of the applied normal force and the applied torque is contemplated. Assume the cutting edge 58, 60 of the blade is divided into number of small elements (N). Each element is assumed to experience orthogonal cutting. The method of calculating the dynamic characteristics of the distal tip at any instant and spatial position on the cutting edge can be developed based on geometric factors. Torque at each instant can be determined by the following equation:

$${T}_{[ total ]} = {\sum }_{f = 1}^{N} [ {F}_{p} , {F}_{n} ( f ( {α}_{d ( i )} , woc ( i ) ) \times r ( i ) ) ]$$

where,
Tₜₒₜₐₗ=total torque applied during penetration of the trocar obturator;
Fₚ=horizontal forces applied during penetration of the trocar obturator;
Fₙ=normal forces applied during penetration of the trocar obturator;
α_{d}=Dynamic Cutting Angle;
woc (i)=width of cutting edge, that is, the length of the cutting edge across the distal tip, which, in accordance with a preferred embodiment of the present invention includes the offset cutting edges and the secondary flat point angle; and
r(i)=radius of element from the axis of the trocar, which varies for each element on the cutting edge.

As those skilled in the art will certainly appreciate, all factors of the preceding equation are substantially predefined with the exception of Tₜₒₜₐₗ and Fₙ. As such, the present invention optimizes these factors to provide a distal tip of a trocar obturator ideally suited for tissue penetration.

Manufacture of this proposed tip design can be accomplished using techniques similar to those employed currently in obturator tip manufacturing. For example, the tip can be manufactured via an injection molding process with the parting line running down the tissue separators and staggering the parting line at the functional tip.

In order to maintain clear visibility, the inside of the tip may have a mating contour similar to the outside in order to maintain a constant wall thickness to prevent visual distortion.

While the preferred embodiments have been shown and described, it will be understood that there is no intent to limit the invention by such disclosure, but rather, is intended to cover all modifications and alternate constructions falling within the spirit and scope of the invention as defined in the appended claims.

1. A trocar obturator, comprising:
a shaft having a proximal end and a distal end;
a tip positioned at the distal end of the shaft, the tip including a distally extending blade structure adapted to reduce penetration forces required during insertion of the trocar obturator;
the blade structure includes a first cutting edge and a second cutting edge.

2. The trocar obturator according to claim 1, wherein the first and second cutting edges are offset.

3. The trocar obturator according to claim 1, wherein a secondary flat point angle is positioned between the first and second cutting edges.

4. The trocar obturator according to claim 1, wherein the first and second cutting edges respectively include a negative cutting angle

5. The trocar obturator according to claim 4, wherein the first and second cutting edges range from −60° and 0°.

6. The trocar obturator according to claim 5, wherein the first and second cutting edges range from −45° and −30°.

7. The trocar obturator according to claim 1, wherein the first and second cutting edges respectively include a positive cutting angle.

8. The trocar obturator according to claim 7, wherein the first and second cutting edges range from 0° to 70°.

9. The trocar obturator according to claim 8, wherein the first and second cutting edges range from 0° to 60°.

10. The trocar obturator according to claim 1, wherein the tip has a generally conical construction.

11. The trocar obturator according to claim 1, wherein the tip has a cone angle of approximately 30° to approximately 150°.

12. A trocar assembly, comprising:
a trocar sleeve and a trocar obturator shaped and dimensioned for movement within the trocar sleeve;
the trocar obturator includes a shaft having a proximal end and a distal end, a tip positioned at the distal end of the shaft, the tip including a distally extending blade structure adapted to reduce penetration forces required during insertion of the trocar obturator, and the blade structure includes a first cutting edge and a second cutting edge.

13. The trocar assembly according to claim 12, wherein the first and second cutting edges are offset.

14. The trocar assembly according to claim 12, wherein a secondary flat point angle is positioned between the first and second cutting edges.

15. The trocar assembly according to claim 12, wherein the first and second cutting edges respectively include a negative cutting angle

16. The trocar assembly according to claim 15, wherein the first and second cutting edges range from −60′ and 0°.

17. The trocar assembly according to claim 12, wherein the first and second cutting edges respectively include a positive cutting angle.

18. The trocar assembly according to claim 17, wherein the first and second cutting edges range from 0° to 70°.

19. The trocar assembly according to claim 12, wherein the tip has a generally conical construction.

20. The trocar assembly according to claim 12, wherein the tip has a cone angle of approximately 30° to approximately 150°.
